(19) United States
(12) Patent Application Publication
(10) Pub. No.: US 2023/0385849 A1
(43) Pub. Date: Nov. 30, 2023

(54) IDENTIFICATION OF FRAUDULENT HEALTHCARE PROVIDERS THROUGH MULTIPRONGED AI MODELING

(71) Applicant: Mastercard International Incorporated, Purchase, NY (US)

(72) Inventors: Athena Stacy-Nieto, Somerville, MA (US); Alok Singh, Gurgaon (IN); Nitish Kumar, Jamshedpur (IN); Kaye Kirschner, New York, NY (US); Mahdi Jadaliha, Wildwood, MO (US); Yuanzheng Du, San Francisco, CA (US); Timothy McBride, Saratoga Springs, UT (US)

(73) Assignee: Mastercard International Incorporated, Purchase, NY (US)

(21) Appl. No.: 17/828,945

(22) Filed: May 31, 2022

(51) Int. Cl. G06Q 30/00; G16H 10/60; G06Q 10/10; G06N 3/08; G06N 3/04

(52) U.S. Cl. CPC G06Q 30/0185; G16H 10/60; G06Q 10/10; G06N 3/088; G06N 3/0454

(57) ABSTRACT

A system and computer-implemented method for identifying fraudulent healthcare providers receives raw claims data from one or more data sources. The raw claims data includes claims associated with a selected healthcare provider. Each of the claims includes one or more claim lines. A first model is executed on the raw claims data. The first model determines a first score for the healthcare provider. A second model is executed on the raw claims data. The second model determines a second score for the healthcare provider. In addition, a third model is executed on the raw claims data. The third model determines a third score for the healthcare provider. A final provider-level risk score is determined for the healthcare provider based on the first, second, and third scores.

### BACKGROUND

The field of the disclosure relates to identifying fraudulent healthcare providers, and more particularly, to identifying fraudulent healthcare providers via electronic health record (EHR) data applied to a multipronged artificial intelligence (AI) model.

Healthcare fraud in the United States results in a massive expense to the nation every year. According to the National Health Care Anti-Fraud Association (NHCAA), in 2018, $3.6 trillion was spent on healthcare in the United States, representing billions of health insurance claims. The NHCAA estimates that the financial losses due to healthcare fraud are in the tens of billions of dollars each year. A conservative estimate is three percent (3%) of total healthcare expenditures, while some government and law enforcement agencies place the loss as high as ten percent (10%) of our annual health outlay, which could mean more than $300 billion. There is a need for improved computer-implemented methods, systems comprising computer-readable media, and electronic devices for identifying fraudulent healthcare providers and detecting claims data anomalies.

### BRIEF DESCRIPTION OF THE DISCLOSURE

This brief description is provided to introduce a selection of concepts in a simplified form that are further described in the detailed description below. This brief description is not intended to identify key features or essential features of the claimed subject matter, nor is it intended to be used to limit the scope of the claimed subject matter. Other aspects and advantages of the present disclosure will be apparent from the following detailed description of the embodiments and the accompanying figures.

In one aspect, a server system is provided. The server system includes a processor and a memory element. The memory element includes computer-executable instructions stored thereon. The computer-executable instructions, when executed by the processor, cause the processor to receive raw claims data from one or more data sources. The raw claims data includes one or more claims associated with a selected healthcare provider. Each of the one or more claims includes one or more claim lines. The processor executes a first model on the raw claims data. The first model determines a first score for the healthcare provider. The processor also executes a second model on the raw claims data. The second model determines a second score for the healthcare provider. Furthermore, the processor executes a third model on the raw claims data. The third model determines a third score for the healthcare provider. Furthermore, the processor determines a final provider-level risk score for the healthcare provider based on the first, second, and third scores.

In another aspect, a computer-implemented method is provided. The method is performed by a server. The method includes receiving raw claims data from one or more data sources. The raw claims data includes one or more claims associated with a selected healthcare provider. Each of the one or more claims includes one or more claim lines. The method also includes executing a first model on the raw claims data and determining, by the first model, a first score for the healthcare provider. In addition, the method includes executing a second model on the raw claims data and determining, by the second model, a second score for the healthcare provider. Furthermore, the method includes executing a third model on the raw claims data and determining, by the third model, a third score for the healthcare provider. The method also includes determining a final provider-level risk score for the healthcare provider based on the first, second, and third scores.

A variety of additional aspects of the claimed subject matter will be set forth in the detailed description that follows. These aspects can relate to individual features of the present disclosure and to combinations of features. Advantages of these and other aspects will become more apparent to those skilled in the art from the following description of the exemplary embodiments which have been shown and described by way of illustration. As will be realized, the present aspects described herein may be capable of other and different aspects, and their details are capable of modification in various respects. Accordingly, the figures and description are to be regarded as illustrative in nature and not as restrictive.

### BRIEF DESCRIPTION OF THE DRAWINGS

The figures described below depict various aspects of systems and methods disclosed therein. It should be understood that each figure depicts an embodiment of a particular aspect of the disclosed systems and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 depicts an exemplary system, in accordance with one or more embodiments of the present invention;

FIG. 2 is an example configuration of a computing device for use in the system shown in FIG. 1;

FIG. 3 is an example configuration of a server for use in the system shown in FIG. 1; and

FIG. 4 is a flowchart illustrating an exemplary computer-implemented method for identifying fraudulent healthcare providers via electronic health record (EHR) data, in accordance with one or more embodiments of the present invention.

Unless otherwise indicated, the figures provided herein are meant to illustrate features of embodiments of this disclosure. These features are believed to be applicable in a wide variety of systems comprising one or more embodiments of this disclosure. As such, the figures are not meant to include all conventional features known by those of ordinary skill in the art to be required for the practice of the embodiments disclosed herein.

### DETAILED DESCRIPTION OF THE DISCLOSURE

The following detailed description of embodiments of the invention references the accompanying figures. The embodiments are intended to describe aspects of the invention in sufficient detail to enable those with ordinary skill in the art to practice the invention. The embodiments of the invention are illustrated by way of example and not by way of limitation. Other embodiments may be utilized, and changes may be made without departing from the scope of the claims. The following description is, therefore, not limiting. The scope of the present invention is defined only by the appended claims, along with the full scope of equivalents to which such claims are entitled.

As used herein, the term “database” includes either a body of data, a relational database management system (RDBMS), or both. As used herein, a database includes, for example, and without limitation, a collection of data including hierarchical databases, relational databases, flat file databases, object-relational databases, object-oriented databases, and any other structured collection of records or data that is stored in a computer system. Examples of RDBMS's include, for example, and without limitation, Oracle® Database (Oracle is a registered trademark of Oracle Corporation, Redwood Shores, Calif.), MySQL, IBM® DB2 (IBM is a registered trademark of International Business Machines Corporation, Armonk, N.Y.), Microsoft® SQL Server (Microsoft is a registered trademark of Microsoft Corporation, Redmond, Wash.), Sybase® (Sybase is a registered trademark of Sybase, Dublin, Calif.), and PostgreSQL. However, any database may be used that enables the systems and methods to operate as described herein.

### Exemplary System

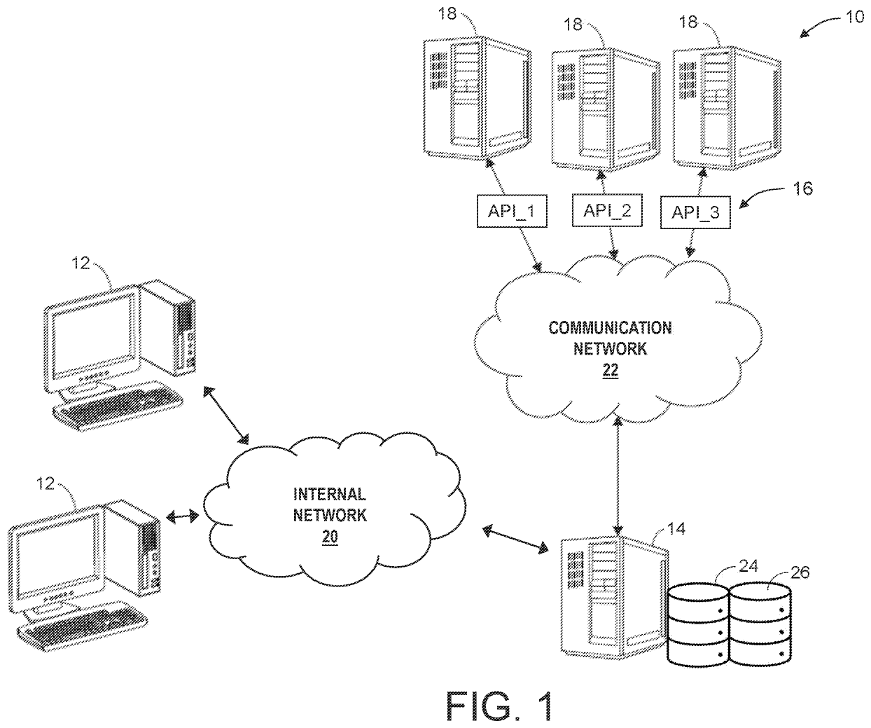

FIG. 1 is a schematic diagram of an exemplary computing environment 10 for identifying fraudulent healthcare providers via electronic health record (EHR) data, according to one aspect of the present invention. In the example embodiment, the environment 10 includes a plurality of computers 12, a server 14 coupled to databases 24 and 26, a plurality of application programming interfaces (APIs) 16, a plurality of data sources 18, an internal network 20, and a communication network 22. The computers 12 and the server 14 may be located within network boundaries of a large organization, such as a corporation, a government office, or the like. The communication network 22 and the APIs 16 may be external to the organization, for example where the APIs 16 are offered by healthcare providers and/or insurance providers or related third parties making healthcare insurance claims data available for analysis, for example, via the data sources 18.

More particularly, the computers 12 and the server 14 may be connected to the internal network 20 of the organization, which may comprise a trusted internal network or the like. Alternatively or in addition, the computers 12 and servers 14 may manage access to the APIs 16 under a common authentication management framework. Each user of a computer 12 may be required to complete an authentication process to access data obtained from the APIs 16 via the server 14. In one or more embodiments, one or more computers 12 may not be internal to the organization but may be permitted access to perform data queries via the common authentication management framework. All or some of the APIs 16 may be maintained and/or owned by the organization and/or may be maintained on the internal network 20 within the scope of the present invention. One of ordinary skill will appreciate that the server 14 may be free of, and/or subject to different protocol(s) of, the common authentication management framework within the scope of the present invention.

Data made available via the APIs 16 may include EHR data comprising medical or healthcare insurance claims data. Further, the server 14 may be maintained by a payment network organization or government organization, and an authenticated employee of the foregoing may access an exemplary system implemented on the server 14 to query the APIs 16 and/or use the obtained information to perform healthcare provider fraud or excessive billing analyses. An employee of the payment network organization or government organization may also access such an exemplary system from a computer 12 to query the APIs 16 and/or use the obtained information to perform healthcare provider fraud or excessive billing analyses. One of ordinary skill will appreciate that embodiments may serve a wide variety of organizations and/or rely on a wide variety of data sources. For example, one or more of the data sources 18 accessed by a system according to embodiments of the present invention may be available to the public. Moreover, one of ordinary skill will appreciate that different combinations of one or more computing devices—including a single computing device or server—may implement the embodiments disclosed herein.

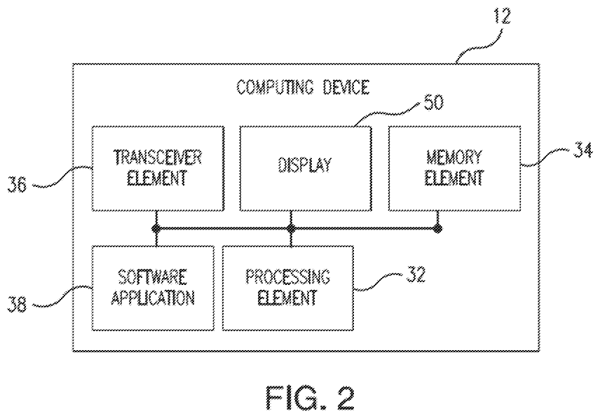

Turning to FIG. 2, it is contemplated that the computers 12 may be workstations. Generally, the computers 12 may include tablet computers, laptop computers, desktop computers, workstation computers, smart phones, smart watches, and the like. In addition, the computers 12 may include copiers, printers, routers, and any other device that can connect to the internal network 20 and/or the communication network 22. Each computer 12 may include a processing element 32 and a memory element 34. Each computer 12 may also include circuitry capable of wired and/or wireless communication with the internal network 20 and/or the communication network 22, including, for example, transceiver elements 36. Further, the computers 12 may respectively include a software application 38 configured with instructions for performing and/or enabling performance of at least some of the steps set forth herein. In one or more embodiments, the software applications 38 comprise programs stored on computer-readable media of memory elements 34. Still further, the computers 12 may respectively include a display 50.

Generally, the server 14 acts as a bridge between the computers 12 and/or internal network 20 of the organization on the one hand, and the communication network 22 and APIs 16 of the outside world on the other hand. In one or more embodiments, the server 14 also provides communication between the computers 12 and internal APIs 16. The server 14 may include a plurality of proxy servers, web servers, communications servers, routers, load balancers, and/or firewall servers, as are commonly known.

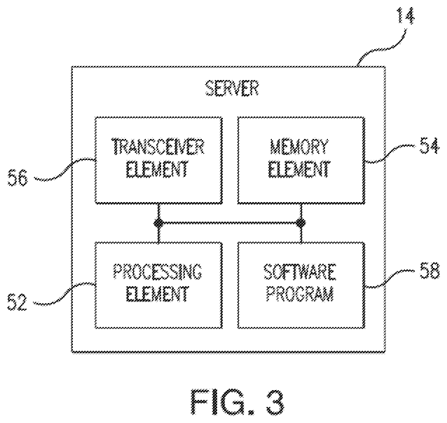

The server 14 also generally implements a platform for managing receipt and storage of claims data (e.g., from APIs 16) and/or performance of requested machine learning or related tasks outlined herein. The server 14 may retain electronic data and may respond to requests to retrieve data as well as to store data. The server 14 may include domain controllers, application servers, database servers, file servers, mail servers, catalog servers or the like, or combinations thereof. In one or more embodiments, one or more APIs 16 may be maintained by the server 14. As depicted in FIG. 3, generally, the server 14 may include a processing element 52, a memory element 54, a transceiver element 56, and a software program 58.

Each API 16 may include and/or provide access to one or more pages or sets of data and/or other content accessed through the communication network 22 (e.g., through the internet) and/or through the internal network 20. Each API 16 may be hosted by or stored on a web server and/or database server, for example. The APIs 16 may include top-level domains such as “.com”, “.org”, “.gov”, and so forth. The APIs 16 may be accessed using software such as a web browser, through execution of one or more script(s) for obtaining EHR data, and/or by other means for interacting with APIs 16 without departing from the spirit of the present invention.

The communication network 22 generally allows communication between the server 14 of the organization and external APIs such as provider APIs 16. The communication network 22 may also generally allow communication between the computers 12 and the server 14, for example, in conjunction with the common authentication framework discussed above and/or secure transmission protocol(s). The internal network 20 may generally allow communication between the computers 12 and the server 14. The internal network 20 may also generally allow communication between the server 14 and internal APIs 16.

The networks 20, 22 may include the internet, cellular communication networks, local area networks, metro area networks, wide area networks, cloud networks, plain old telephone service (POTS) networks, and the like, or combinations thereof. The networks 20, 22 may be wired, wireless, or combinations thereof and may include components such as modems, gateways, switches, routers, hubs, access points, repeaters, towers, and the like. The computers 12, server 14, and/or APIs 16 may, for example, connect to the networks 20, 22 either through wires, such as electrical cables or fiber optic cables, or wirelessly, such as RF communication using wireless standards such as cellular 2G, 3G, 4G or 5G, Institute of Electrical and Electronics Engineers (IEEE) 802.11 standards such as WiFi, IEEE 802.16 standards such as WiMAX, Bluetooth™, or combinations thereof.

The transceiver elements 36, 56 generally allow communication between the computers 12, the server 14, the networks 20, 22, and/or the APIs 16. The transceiver elements 36, 56 may include signal or data transmitting and receiving circuits, such as antennas, amplifiers, filters, mixers, oscillators, digital signal processors (DSPs), and the like. The transceiver elements 36, 56 may establish communication wirelessly by utilizing radio frequency (RF) signals and/or data that comply with communication standards such as cellular 2G, 3G, 4G or 5G, Institute of Electrical and Electronics Engineers (IEEE) 802.11 standard such as WiFi, IEEE 802.16 standard such as WiMAX, Bluetooth™, or combinations thereof. In addition, the transceiver elements 36, 56 may utilize communication standards such as ANT, ANT+, Bluetooth™ low energy (BLE), the industrial, scientific, and medical (ISM) band at 2.4 gigahertz (GHz), or the like. Alternatively, or in addition, the transceiver elements 36, 56 may establish communication through connectors or couplers that receive metal conductor wires or cables, like Cat 6 or coax cable, which are compatible with networking technologies such as ethernet. In certain embodiments, the transceiver elements 36, 56 may also couple with optical fiber cables. The transceiver elements 36, 56 may respectively be in communication with the processing elements 32, 52 and/or the memory elements 34, 54.

The memory elements 34, 54 may include electronic hardware data storage components such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, universal serial bus (USB) drives, or the like, or combinations thereof. In some embodiments, the memory elements 34, 54 may be embedded in, or packaged in the same package as, the processing elements 32, 52. The memory elements 34, 54 may include, or may constitute, a “computer-readable medium.” The memory elements 34, 54 may store the computer-executable instructions, code, code segments, software, firmware, programs, applications, apps, services, daemons, or the like that are executed by the processing elements 32, 52. In one or more embodiments, the memory elements 34, 54 respectively store the software applications/program 38, 58. The memory elements 34, 54 may also store settings, data, documents, sound files, photographs, movies, images, databases, and the like.

The processing elements 32, 52 may include electronic hardware components such as processors. The processing elements 32, 52 may include microprocessors (single-core and multi-core), microcontrollers, digital signal processors (DSPs), field-programmable gate arrays (FPGAs), analog and/or digital application-specific integrated circuits (ASICs), or the like, or combinations thereof. The processing elements 32, 52 may include digital processing unit(s). The processing elements 32, 52 may generally execute, process, or run computer-executable instructions, code, code segments, software, firmware, programs, applications, apps, processes, services, daemons, or the like. For instance, the processing elements 32, 52 may respectively execute the software applications/program 38, 58. The processing elements 32, 52 may also include hardware components such as finite-state machines, sequential and combinational logic, and other electronic circuits that can perform the functions necessary for the operation of the current invention. The processing elements 32, 52 may be in communication with the other electronic components through serial or parallel links that include universal busses, address busses, data busses, control lines, and the like.

Referring back to FIG. 1, the server 14 may manage queries to, and responsive EHR data received from, APIs 16, and perform related analytical functions (e.g., as requested by one or more of the computers 12) in accordance with the description set forth herein. In one or more embodiments, the EHR data may be acquired by other means, and the steps for analysis laid out herein may be requested and/or performed by different computing devices (or by a single computing device), without departing from the spirit of the present invention.

The EHR data may be stored in databases, such as the databases 24, 26, managed by the server 14 utilizing any of a variety of formats and structures within the scope of the invention. For instance, relational databases and/or object-oriented databases may embody the databases 24, 26. Similarly, the APIs 16 and/or databases 24, 26 may utilize a variety of formats and structures within the scope of the invention, such as Simple Object Access Protocol (SOAP), Remote Procedure Call (RPC), and/or Representational State Transfer (REST) types. One of ordinary skill will appreciate that—while examples presented herein may discuss specific types of databases—a wide variety may be used alone or in combination within the scope of the present invention.

Through hardware, software, firmware, or various combinations thereof, the processing elements 32, 52 may—alone or in combination with other processing elements—be configured to perform the operations of embodiments of the present invention. Specific embodiments of the technology will now be described in connection with the attached drawing figures. The embodiments are intended to describe aspects of the invention in sufficient detail to enable those skilled in the art to practice the invention. Other embodiments can be utilized and changes can be made without departing from the scope of the present invention. The system may include additional, less, or alternate functionality and/or device(s), including those discussed elsewhere herein. The following detailed description is, therefore, not to be taken in a limiting sense. The scope of the present invention is defined only by the appended claims, along with the full scope of equivalents to which such claims are entitled.

### Exemplary Computer-Implemented Methods

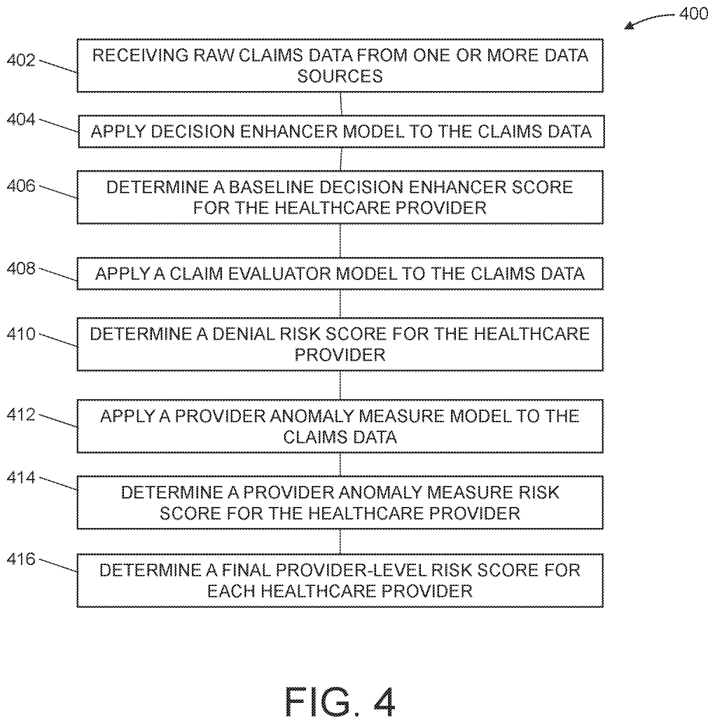

FIG. 4 is a flowchart illustrating an exemplary computer-implemented method 400 for identifying fraudulent healthcare providers via electronic health record (EHR) data, in accordance with one embodiment of the present disclosure. The operations described herein may be performed in the order shown in FIG. 4 or, according to certain inventive aspects, may be performed in a different order. Furthermore, some operations may be performed concurrently as opposed to sequentially, and/or some operations may be optional, unless expressly stated otherwise or as may be readily understood by one of ordinary skill in the art.

The computer-implemented method 400 is described below, for ease of reference, as being executed by exemplary devices and components introduced with the embodiments illustrated in FIGS. 1-3. In one embodiment, the computer-implemented method 400 is implemented by the server 14. In the exemplary embodiment, the computer-implemented method 400 relates to applying a multipronged artificial intelligence (AI) model to claims data submitted by one or more healthcare providers to identify fraudulent healthcare providers. While operations within the computer-implemented method 400 are described below regarding the server 14, according to some aspects of the present invention, the computer-implemented method 400 may be implemented using any other computing devices and/or systems through the utilization of processors, transceivers, hardware, software, firmware, or combinations thereof. A person having ordinary skill will also appreciate that responsibility for all or some of such actions may be distributed differently among such devices or other computing devices without departing from the spirit of the present disclosure.

One or more computer-readable medium(s) may also be provided. The computer-readable medium(s) may include one or more executable programs stored thereon, wherein the program(s) instruct one or more processors or processing units to perform all or certain of the steps outlined herein. The program(s) stored on the computer-readable medium(s) may instruct the processor or processing units to perform additional, fewer, or alternative actions, including those discussed elsewhere herein.

At operation 402, raw claims data from one or more data sources, such as the data sources 18 (shown in FIG. 1), are received by the server 14 (shown in FIG. 1). The data include, for example, data corresponding to a plurality of claims data of a plurality of healthcare providers. Operation 402 may be executed by one or both of a computing device and a server. The claims data may be obtained periodically, continuously, and/or upon request from a variety of sources. For example, an automated data acquisition process may cause intermittent batch downloads of claims data from APIs associated with healthcare service providers and/or third-party databases storing such data to network servers and/or computing devices. It should be noted that the frequencies discussed above can be any determined frequency that enables the method 400 to function as described herein.

The raw claims data or provider data may be extracted from tabulated claims data regarding, for example, inpatient and/or outpatient medical insurance claims submitted by the plurality of providers. In one or more embodiments, the plurality of providers may be selected according to, for example, specialty, size, geographic location, or other selection criteria. The selection criteria may be determined at least in part based on observing the impact of various combinations of criteria on accuracy of fraudulent billing predictions using the multipronged AI model described herein.

At operation 404, a decision enhancer model is applied to the raw claims data. In particular, in the exemplary embodiment, the processing element 52 executes the decision enhancer model on the raw claims data. In the example embodiment, the decision enhancer model is a rules-based claim line editing model of the multipronged AI model. The decision enhancer model takes the healthcare claims data as input and provides a score for each provider's risk of fraudulent or otherwise problematic behavior. In one embodiment, the decision enhancer is a SQL-based model to score a healthcare provider's risk of fraudulent or otherwise problematic behavior. The SQL scripts can be run on any database of healthcare claims, and the general scoring method can be translated to other programming languages. In particular, the decision enhancer model implements “hard” claims editing rules devised by the Center for Medicare and Medicaid Services (CMS), including a collection of National Correct Coding Initiative (NCCI) edits. The hard rules detect problems with claims such as medically unlikely charges (e.g., charging a patient for the removal of three tonsils), mutually exclusive medical procedures performed on the same patient, etc. A non-exhaustive list of example hard rules is outlined in the following Table 1. Note that as used in Table 1, “(fac)” refers to UB-04 facilities claims and “(phys)” refers to professional physical claims (e.g. forms CMS-1500 or 837-P).

TABLE 1

| RULE NAME | RULE DESCRIPTION |
|---|---|
| 26_rev | A CPT code was billed with both modifier 26 (professional services) |
| | and a revenue code |
| add_on | Add-on procedure was applied to an incorrect initial procedure |
| age_diag | Patient too old or young for diagnosis |
| age_proc | Patient too old or young for procedure |
| alf_pos | Assisted Living Facility visits with a bad Place of Service (POS) code |
| amb_mod | Ambulance ride without cpt modifier |
| amb_only | Ambulance ride without attending medical claim |
| anes_p | Anesthesia service included in surgical procedure (phys) |
| cms_man_f | CPT Manual or CMS manual coding instructions (fac) |
| cms_man_p | CPT Manual or CMS manual coding instructions (phys) |
| code_mis_f | Misuse of column two code with column one code (fac) |
| code_mis_p | Misuse of column two code with column one code (phys) |
| cpt_def_f | HCPCS/CPT procedure code definition (fac) |
| cpt_def_p | HCPCS/CPT procedure code definition (phys) |
| drug_proc | Administered Drugs Without Detailed Coding |
| dup_phys | Duplicate Physician Billed Procedures - same physician billed a |
| | patient twice |
| em_mod | An Evaluation and Management code reported with modifier −26 |
| | or −TC |
| em_pos | An Evaluation and Management code reported with an invalid Place |
| | of Service (POS) code |
| finger | A procedure performed on a finger was billed without a modifier to |
| | identify which finger |
| gender_f | Gender-specific (formerly Designation of sex) procedures (phys) |
| in_pos | An inpatient consult was billed with an incorrect Place of Service |
| | (POS) code |
| inv_pd | Invalid Primary Diagnosis |
| lab_pan_f | Laboratory panel (fac) |
| lab_pan_p | Laboratory panel (phys) |
| more_ext_f | More extensive procedure (fac) |
| more_ext_p | More extensive procedure (phys) |
| mue | Excessive units charged to the patient within one day for the given |
| | procedure code |
| mut_exc_f | Mutually exclusive procedures (fac) |
| mut_exc_p | Mutually exclusive procedures (phys) |
| no_pos | Professional service without POS code |
| no_prim | Claim without primary diagnosis |
| no_proc | Professional service without procedure code |
| off_pos | Office visit claim with unrelated Place of Service (POS) code |
| out_pos | An outpatient consult was billed with an incorrect Place of Service |
| | (POS) code |
| sep_proc_f | CPT “separate procedure” definition (fac) |
| sep_proc_p | CPT “separate procedure” definition (phys) |
| seq_proc_p | Sequential procedure (phys) |
| snf_pos | Skilled Nursing Facility visits with a bad Place of Service (POS) code |
| stand_prep_p | Standard preparation/monitoring services for anesthesia (phys) |
| stand_surg_f | Standards of medical/surgical practice (fac) |
| stand_surg_p | Standards of medical/surgical practice (phys) |
| toe | A procedure performed on a toe was billed without a modifier to |
| | identify which toe |

The hard rules are also combined with “soft” rules that are not explicitly spelled out by CMS, such as upcoding (charging for a more expensive procedure than was rendered), lab unbundling (charging for several smaller individual procedures instead of a single all-inclusive bundled procedure), etc. A non-exhaustive list of example soft rules is outlined in the following Table 2:

TABLE 2

| RULE NAME | RULE DESCRIPTION |
|---|---|
| em_con | Ratio of high-level (99244, 99245) to medium-level codes |
| | (99243) over last 30 days for consultations was twice the peer |
| | average |
| em_est | Ratio of high-level (99214, 99215) to medium-level codes |
| | (99213) over last 30 days for established patients was twice |
| | the peer average |
| em_new | Ratio of high-level (99204, 99205) to medium-level codes |
| | (99203) over last 30 days for new patients was twice the peer |
| | average |
| em_z00 | Provider billed complex E&M codes with Z00 diagnosis |
| | within past 30 days |
| evisits | Patient had both an e-visit and an office visit within past 7 |
| | days |
| iron_man | Patient had over 5 orthotic procedures done over past 90 days |
| iv_jcode | Patient had IV code performed without corresponding J-code |
| lab_acutehep | Patient had no acute hepatitis lab panel but over 2 lab panel |
| | components performed within last 7 days |
| lab_elec | Patient had no electrolyte lab panel but over 2 lab panel |
| | components performed within last 7 days |
| lab_genh | Patient had no general health lab panel but over 4 lab panel |
| | components performed within last 7 days |
| lab_gluc | Patient had multiple glucose labs (83063, 83036, 83037) |
| | performed within an 80-day period |
| lab_hep | Patient had no hepatic function lab panel but over 4 lab panel |
| | components performed within last 7 days |
| lab_lipid | Patient had no lipid lab panel but more than one lab panel |
| | components performed within last 7 days |
| lab_metab | Patient had no metabolic lab panel but over 5 lab panel |
| | components performed within last 7 days |
| lab_metcomp | Patient had no comprehensive metabolic lab panel but over 8 |
| | lab panel components performed within last 7 days |
| lab_obst | Patient had no obstetric lab panel but over 5 lab panel |
| | components performed within last 7 days |
| lab_renal | Patient had no renal function lab panel but over 7 lab panel |
| | components performed within last 7 days |
| lab_unbundle_prov | Provider billed more than ten times as many lab component |
| | procedure codes than lab panel procedure codes over past 30 |
| | days |

For each claim line, the decision enhancer model determines whether the claim line violates one or more of the hard and soft rules. That is, the decision enhancer model identifies each claim line that violates one or more of the hard and soft rules. The decision enhancer model then flags each claim line that violates one or more of the above rules. The rate at which the problematic behavior occurs is aggregated over various pre-defined moving time windows for each provider using either the claim service date or claim submission date. The time windows may include, for example, a rolling thirty (30) day window and a rolling ninety (90) day window. It is contemplated that other rolling time windows may be selected, as desired. The time-window aggregates, called “provider profiling counters,” are compared with peers in the provider's specialty (e.g., cardiology, pediatrics, etc.).

At operation 406, a decision enhancer score is determined for each healthcare provider based on the healthcare provider's raw claims data. Healthcare providers that break the hard and/or soft rules at increased rates, as compared to its peers, are given a higher decision enhancer score. Thus, a healthcare provider that unintentionally makes small numbers of claims errors will be considered lower risk than a healthcare provider that makes a large number of such errors, which may be more indicative of fraudulent intent.

In the example embodiment, the formula for the baseline decision enhancer score over a ninety (90) day time window is as follows:

$$de\_score \_base = \frac{( total flagged billed amount over past 90 days )}{( total billed amount over past 90 days )}$$

The ninety (90) day time window may be adjusted, and a weighting factor by specialty may be added as well. An example weighting formula is as follows:

$$weighting = \frac{( average de\_score \_base for all providers in dataset )}{( average de\_score \_base for provider ’ s specialty )}$$

As described further below, the de score can be combined with scores from other models to provide a provider-level risk score that provides a more comprehensive view of the healthcare provider's overall behavior and risk. The decision enhancer score can be used to flag healthcare providers for further investigation by healthcare fraud experts. The rate at which a healthcare provider is flagged for breaking a particular rule can also be used as a reason code to guide investigators towards what to look for when examining a particular healthcare provider. For each healthcare provider, the decision enhancer flags are marked for the specific problematic claim lines on which an investigator should focus.

At operation 408, a trained claim evaluator model is applied to the raw claims data. In particular, in the exemplary embodiment, the processing element 52 executes the trained claim evaluator model on the raw claims data. A diagnosis and procedure code for each claim line is submitted to the claim evaluator model. In the example embodiment, the claim evaluator model may be applied substantially simultaneously with the decisions enhancer model or may be applied in a serial manner. The decision enhancer model and the claim evaluator model are not mutually exclusive. Rather, each is applied to the raw claims data independently of the other.

In the example embodiment, the trained claim evaluator model employs a neural network algorithm. The claim evaluator model is a supervised machine learning component of the multipronged AI model used to predict whether a selected claim will be denied or approved. The claim evaluator model provides a “denial risk” score for each claim processed by the model. The denial risk score is based on a combination of an output from the neural network algorithm and a relevancy index score.

After the model evaluates all claims on a claim-by-claim basis, at operation 410, claim-line level denial risk scores are aggregated to the healthcare provider level to determine “denial risk” score for the healthcare provider, such that healthcare providers predicted to have a high rate of claim denials are given higher denial risk scores.

The type of supervised training data used to train the claim evaluator model includes, for example, historic claims data. In examples, the training data should comprise a wide number of different claims from a plurality of healthcare providers which are known to be related to an activity of interest (such as fraudulent activity, for example). The historic claims data may include data which has been constructed based on labelled claims categories (e.g., specialty) and observations collected from insurance companies' approval or denial decisions, for example. It is contemplated that the historical data may be labelled based on any other selection criteria that enables the claim evaluator model to function as described herein.

The claim evaluator model to be trained (such as a neural network algorithm) may be configured to use the training examples provided in the training data during a training phase in order to learn how to identify instances of fraudulent claims activity and/or otherwise denied claims.

In a specific example of a neural network, the neural network may be constructed of an input layer and an output layer, with a number of ‘hidden’ layers therebetween. Each of these layers may include a number of distinct nodes. The nodes of the input layer are each connected to the nodes of the first hidden layer. The nodes of the first hidden layer are then connected to the nodes of the following hidden layer or, in the event that there are no further hidden layers, the output layer. However, while, in this specific example, the nodes of the input layer are described as each being connected to the nodes of the first hidden layer, it will be appreciated that the present disclosure is not particularly limited in this regard. Indeed, other types of neural networks may be used in accordance with embodiments of the disclosure as desired depending on the situation to which embodiments of the disclosure are applied.

The nodes of the neural network each take a number of inputs and produce an output based on those inputs. The inputs of each node have individual weights applied to them. The inputs (such as the properties of the accounts) are then processed by the hidden layers using weights, which are adjusted during training. The output layer produces a prediction from the neural network (which varies depending on the input that was provided).

In examples, during training, adjustment of the weights of the nodes of the neural network is achieved through linear regression models. However, in other examples, logistic regression can be used during training. Basically, training of the neural network is achieved by adjusting the weights of the nodes of the neural network in order to identify the weighting factors which, for the training input data provided, produce the best match to the actual data which has been provided.

In other words, during training, both the inputs and target outputs of the neural network may be provided to the model to be trained. The model then processes the inputs and compares the resulting output against the target data (i.e., sets of claims data from healthcare providers messages and/or individual accounts which are known to include denied claims). Differences between the output and the target data are then propagated back through the neural network, causing the neural network to adjust the weights of the respective nodes of the neural network. However, in other examples, training can be achieved without the outputs, using constraints of the system during the optimization process.

Once trained, new input data (i.e., new claims data from healthcare providers) can then be provided to the input layer of the trained claim evaluator model, which will cause the trained claim evaluator model to generate (on the basis of the weights applied to each of the nodes of the neural network during training) a predicted output for the given input data (being a prediction of the claims which are likely to be denied, for example, by being linked to fraudulent activity).

However, it will be appreciated that the neural network described here is not particularly limiting to the present disclosure. More generally, any type of machine learning model or machine learning algorithm can be used in accordance with embodiments of the disclosure.

The relevancy index is determined based on the same historic claims data used to train the neural network algorithm. The relevancy index score provides an indication of whether the provided medical services (based on the procedure code of the claim, for example) are relevant to the recorded diagnosis of the claim. Thus, the relevancy index score is indicative of the number of times a certain medical service is associated with a certain diagnosis in historic claims data. The relevancy index may consider the number of providers that provide such a service/diagnosis pairing, the overall number of claims that include such as pairing, and the number of patients that include such a pairing. Thus, if a certain service/diagnosis pairing shows up in claims data for a certain provider, but not other providers, the relevancy index score would indicate that such a pairing is likely indicative of fraudulent activity by the healthcare provider.

At operation 412, a provider anomaly measure model is applied to the raw claims data. In particular, in the exemplary embodiment, the processing element 52 executes the provider anomaly measure model on the raw claims data. In the example, the provider anomaly measure model is an unsupervised machine learning component of the multipronged AI model that provides a measure of how anomalous a healthcare provider is relative to its specialty peer group(s). The provider anomaly measure model utilizes isolation forest and autoencoder based anomaly detection algorithms, as described herein.

At operation 414, the provider anomaly measure model determines a provider anomaly measure risk score for a selected healthcare provider based on that healthcare provider's raw claims data. In particular, the provider anomaly measure model includes three (3) separate anomaly detection models, the outputs of which are combined to determine the provider anomaly measure risk score. The provider anomaly measure model includes autoencoder-based anomaly detection, which is a deep learning method that utilizes an encoder-decoder architecture for detecting anomalies in the claims data. The provider anomaly measure model also includes an isolation forest machine learning model that detects anomalies based on outlier detection (how easily a point can be removed from the population). In addition, the provider anomaly measure model includes generative adversarial network (GAN) based anomaly detection.

In the example embodiment, healthcare claims data is provided by various data sources based on bill type (e.g., pharmacy data, physician data, etc.). The entities involved in a healthcare claim, such as the healthcare provider and patient, are associated with secondary data such as patient enrollment data and demographic data. Extensive feature engineering is performed on historic claims data to generate meaningful variables for use in the provider anomaly measure model. For example, and without limitation, such modelling variables may include overall utilization variables (e.g., billed amount, paid services at patient and claim level, claims per patient, etc.), velocity variables (e.g., velocity at patient, claims, and service line level), and domain specific variables (e.g., procedure and diagnosis codes categorization into broader categories, comorbid conditions, etc.). The features generated at the claim level are aggregated at the provider level and used along with provider level features to be fed to the three (3) anomaly detection methods describe above. The outcome of each anomaly detection method is aggregated to generate the final provider anomaly measure risk score.

To validate and substantiate the provider anomaly measure risk scores generated by the provider anomaly measure model, the healthcare providers are profiled on indexes generated using algorithms inspired from Natural Language Processing (NLP), which includes network analysis and statistical methods that can capture existing patterns prevalent in healthcare claims fraud. The provider anomaly measure risk scores are validated based on the extent to which the scores hold the hypothesis of the generated indexes.

In one embodiment, a phantom billing index is used. The phantom billing index identifies excessive and unnecessary services provided to patients. Such services are identified by segmenting frequently co-occurring diagnosis and procedures in a healthcare provider's claims. To generate the co-occurring clusters of diagnosis and procedures, embeddings generated from NLP-based methods are used. In the example embodiment, sequence to sequence (seq2seq) models are used to generate procedure and diagnosis representation based on their occurrence in the claims data. Sequences of diagnosis codes and procedure codes are created at the claim level. Because there is no inherent order in the sequence of procedure and diagnosis in a claim, procedure and diagnosis codes are randomly permuted to generate multiple sequences. The healthcare provider identifier (ID) is distributed in between every two (2) codes to form the final sequences. The provider ID is distributed in such a way that a seq2seq model can capture provider ID and the corresponding diagnosis codes and procedure codes in the same context window and learn through cross entity interaction. The sequences are passed through a word2vec model to get procedure and diagnosis representation in the same embedding space. The representations obtained are segmented together to get the clusters of co-occurring procedure codes and diagnosis codes. Healthcare providers having diagnosis codes and procedure codes from a large number of clusters can be indicative of unnecessary or spurious services being provided.

In an embodiment, a suspicious communities may be used as an index. The provider anomaly measure model may use network analysis on a provider-to-provider referral graph. Community detection may be applied on the graph to identify several small communities of healthcare providers that collude among themselves and participate in fraudulent behavior. Graph techniques can capture information such as a nexus between healthcare providers referring to each other. Often these healthcare providers are responsible for committing institutional large-scale fraud. In provider referral representation, the provider anomaly measure model captures the provider-to-provider relationship in a homogeneous provider referral graph with nodes as healthcare providers. Edges between two (2) healthcare providers exist if the same patient visits the two (2) healthcare providers within a thirty (30) day period. The edges act as a proxy for provider-to-provider referral due to the general unavailability of referral information. It is noted that, to obtain suspicious communities on the graph, any community detection method may be employed that enables the provider anomaly measure model to function as described herein.

In an example, provider entropy may be used as another index. The provider anomaly measure model may implement statistical methods to measure a skewness in a healthcare provider's revenue by looking at how the provider is charging patients and claims. For example, a provider entropy index represents how evenly a healthcare provider bills each claim. A low entropy value is indicative of a skewed billed amount per claim (e.g., where a provider bills one patient a substantially increased amount as compared to other patients for the same service). For example, in kickback situations, a healthcare provider will have a highly skewed billing distribution due to a nexus with the patient, and hence, have a decreased entropy value. The provider entropy index uncovers patterns where a healthcare provider and patient form a nexus amongst themselves and tend to participate in fraudulent activities.

At operation 416, the server 14 determines a final provider-level risk score for each healthcare provider based on its baseline decision enhancer score, denial risk score, and provider anomaly measure risk score. In the example embodiment, the final provider-level risk score is a simple average of the three (3) scores, as determined by the respective models described above. However, it is contemplated that the three (3) scores may be combined using any number of techniques to arrive at a final provider-level risk score. For example, in certain embodiments, each of the baseline decision enhancer score, denial risk score, and provider anomaly measure risk score may be weighted differently and combined using techniques that account for weighting factors.

The systems and methods described herein provide for identifying fraudulent healthcare providers via electronic health record (EHR) data. In particular, a multipronged AI model is applied to a healthcare provider's claims data to determine various scores that can be utilized to calculate an overall provider-level risk score. Provider profiling counters allow an investigator to quickly visualize an aggregate of long-term provider activity without having to slowly sift through large numbers of claims. The adjustable provider profiling counters allow for faster processing of claims data, thereby increasing the efficiency of a computer processing the claims data. Further, the systems and methods described generate the claim-level scores as well as the provider-level risk score, and flag specific claims that are identified as problematic at a claim line level. This facilitates efficient claims investigation by narrowing the focus of an investigator to specific problematic claim lines.

### Additional Considerations

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, it should be understood that the legal scope of the description is defined by the words of the claims and equivalent language. The detailed description is to be construed as exemplary only and does not describe every possible embodiment because describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order recited or illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. The foregoing statements in this paragraph shall apply unless so stated in the description and/or except as will be readily apparent to those skilled in the art from the description.

Certain embodiments are described herein as including logic or a number of routines, subroutines, applications, or computer-executable instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processor, may be implemented as special purpose or as general purpose. For example, the processor may comprise dedicated circuitry or logic that is permanently configured, such as an application-specific integrated circuit (ASIC), or indefinitely configured, such as a field-programmable gate array (FPGA), to perform certain operations. The processor may also comprise programmable logic or circuitry (e.g., as encompassed within a general-purpose processor or other programmable processor) that is temporarily configured by software to perform certain operations. It will be appreciated that the decision to implement the processor as special purpose, in dedicated and permanently configured circuitry, or as general purpose (e.g., configured by software) may be driven by cost and time considerations.

Accordingly, the term “processor” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processor is temporarily configured (e.g., programmed), each of the processors need not be configured or instantiated at any one instance in time. For example, where the processor comprises a general-purpose processor configured using software, the general-purpose processor may be configured as respective different processors at different times. Software may accordingly configure the processor to constitute a particular hardware configuration at one instance of time and to constitute a different hardware configuration at a different instance of time.

Computer hardware components, such as transceiver elements, memory elements, processors, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, at a later time, access the memory device to retrieve and process the stored output. Computer hardware components may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processors may be located in a single location (e.g., within a home environment, an office environment or as a server farm), while in other embodiments the processors may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processor and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

Although the disclosure has been described with reference to the embodiments illustrated in the attached figures, it is noted that equivalents may be employed, and substitutions made herein, without departing from the scope of the disclosure as recited in the claims.

Having thus described various embodiments of the disclosure, what is claimed as new and desired to be protected by Letters Patent includes the following:

What is claimed is:

1. A server system comprising:
a processor; and
a memory element comprising computer-executable instructions stored thereon, the computer-executable instructions, when executed by the processor, cause the processor to:
receive raw claims data from one or more data sources, the raw claims data including one or more claims associated with a selected healthcare provider, each of the one or more claims including one or more claim lines;
execute a first model on the raw claims data;
determine, by the first model, a first score for the healthcare provider;
execute a second model on the raw claims data;
determine, by the second model, a second score for the healthcare provider;
execute a third model on the raw claims data;
determine, by the third model, a third score for the healthcare provider; and
determine a final provider-level risk score for the healthcare provider based on the first, second, and third scores.

2. The server system in accordance with claim 1,
said first model comprising a SQL-based model that implements a first set of rules devised by the Center for Medicare and Medicaid Services (CMS) and a second set of rules that are not explicitly devised by CMS.

3. The server system in accordance with claim 2,
said computer-executable instructions, when executed by the processor, further causing the processor to:
identify, by the first model, each claim line that violates one or more of the following: one or more of the first set of rules and one or more of the second set of rules; and
flag each identified claim to generate one or more flagged claims.

4. The server system in accordance with claim 3, wherein the first score for the healthcare provider is determined by dividing a total billed amount for the one or more flagged claims in a provider profiling counter by a total billed amount of all claims in the provider profiling counter, the provider profiling counter comprising a pre-defined moving time window.

5. The server system in accordance with claim 1,
said second model comprising a neural network algorithm.

6. The server system in accordance with claim 5, wherein the second model is trained using supervised training data including historic claims data, the historic claims data including data that has been constructed based on labelled claims categories and claim decision outcomes.

7. The server system in accordance with claim 5, wherein the second score is based on a combination of an output from the neural network algorithm and a relevancy index score.

8. The server system in accordance with claim 7,
said relevancy index score being indicative of whether a procedure code of a respective claim is relevant to a diagnosis of the respective claim.

9. The server system in accordance with claim 1,
said third model comprising three separate anomaly detection models,
wherein outputs of the three separate anomaly detection models are combined to determine the third score.

10. The server system in accordance with claim 9, wherein the three separate anomaly detection models include the following:
an autoencoder-based anomaly detection model that utilizes an encoder-decoder architecture to detect anomalies in the raw claims data;
an isolation forest machine learning model that detects anomalies in the raw claims data based on outlier detection; and
a generative adversarial network (GAN) based anomaly detection model.

11. A computer-implemented method performed by a server, the method comprising:
receiving raw claims data from one or more data sources, the raw claims data including one or more claims associated with a selected healthcare provider, each of the one or more claims including one or more claim lines;
executing a first model on the raw claims data;
determining, by the first model, a first score for the healthcare provider;
executing a second model on the raw claims data;
determining, by the second model, a second score for the healthcare provider;
executing a third model on the raw claims data;
determining, by the third model, a third score for the healthcare provider; and
determining a final provider-level risk score for the healthcare provider based on the first, second, and third scores.

12. The computer-implemented method in accordance with claim 11,
said first model comprising a SQL-based model that implements a first set of rules devised by the Center for Medicare and Medicaid Services (CMS) and a second set of rules that are not explicitly devised by CMS.

13. The computer-implemented method in accordance with claim 12, further comprising:
identifying, by the first model, each claim line that violates one or more of the following:
one or more of the first set of rules and one or more of the second set of rules; and flagging each identified claim to generate one or more flagged claims.

14. The computer-implemented method in accordance with claim 13, wherein the first score for the healthcare provider is determined by dividing a total billed amount for the one or more flagged claims in a provider profiling counter by a total billed amount of all claims in the provider profiling counter, the provider profiling counter comprising a pre-defined moving time window.

15. The computer-implemented method in accordance with claim 11,
said second model comprising a neural network algorithm.

16. The computer-implemented method in accordance with claim 15, wherein the second model is trained using supervised training data including historic claims data, the historic claims data including data that has been constructed based on labelled claims categories and claim decision outcomes.

17. The computer-implemented method in accordance with claim 15, wherein the second score is based on a combination of an output from the neural network algorithm and a relevancy index score.

18. The computer-implemented method in accordance with claim 17,
said relevancy index score being indicative of whether a procedure code of a respective claim is relevant to a diagnosis of the respective claim.

19. The computer-implemented method in accordance with claim 11,
said third model comprising three separate anomaly detection models,
wherein outputs of the three separate anomaly detection models are combined to determine the third score.

20. The computer-implemented method in accordance with claim 19, wherein the three separate anomaly detection models include the following:
an autoencoder-based anomaly detection model that utilizes an encoder-decoder architecture to detect anomalies in the raw claims data;
an isolation forest machine learning model that detects anomalies in the raw claims data based on outlier detection; and
a generative adversarial network (GAN) based anomaly detection model.
